(19) United States
(12) Patent Application Publication
(10) Pub. No.: US 2006/0204598 A1
(43) Pub. Date: Sep. 14, 2006

(54) NICOTINE-ALTERNATIVE COMPOSITIONS AND METHODS OF PRODUCING SUCH COMPOSITIONS

(76) Inventor: Marshall Anlauf Thompson, Camarillo, CA (US)

Correspondence Address: KELLY LOWRY & KELLEY, LLP, 6320 CANOGA AVENUE, SUITE 1650, WOODLAND HILLS, CA, 91367, US

(21) Appl. No.: 11/420,426

(22) Filed: May 25, 2006

Related U.S. Application Data
(63) Continuation-in-part of application No. 10/216,023, filed on Aug. 9, 2002, now abandoned.
(63) Continuation-in-part of application No. 10/015,101, filed on Dec. 10, 2001, now abandoned.

(51) Int. Cl. A61K 36/81

(52) U.S. Cl. 424/751

(57) ABSTRACT

A method for producing a consumable nicotine-alternative composition includes measuring a quantity of a cigarette nicotine-alternative alkaloid and/or a larger quantity of lobeline. The quantities of cigarette nicotine-alternative alkaloid and/or lobeline are diluted into one or more successive intermediate solutions, a last of which constitutes a final solution. The final solution is apportioned so that each portion contains a precise quantity of cigarette nicotine-alternative alkaloid and/or lobeline appropriate for consumption in a single use by a single person. Each portion is introduced into a separate single-serving dispenser.

### BACKGROUND OF THE INVENTION

The present invention relates generally to nicotine or nicotine-alternative beverages that serve as cigarette substitutes for individuals attempting to quit smoking. More particularly, the present invention relates to a method for producing a consumable composition having a precise, single-serving quantity of cigarette nicotine-alternative using conventional beverage production equipment.

When the cigarette-rolling machine was patented in 1881, it enabled a relatively minor product of the time, the cigarette, to become an extremely popular product. It increased cigarette production by 5000 percent and cut the price of an individual cigarette in half. However, the increase in the cigarette's popularity also brought with it a host of health-related problems.

Nicotine is the mechanism by which tobacco dependence is formed. Nicotine is an organic compound, an alkaloid found naturally in the nightshade family of plants, such as tobacco and tomatoes. Nicotine constitutes 0.3 to 5% of the tobacco plant by dry weight, with biosynthesis taking place in the roots, and accumulates in the leaves. Nicotine is a potent nerve poison and is included in many insecticides. In lower concentrations, the substance is a stimulant and is one of the main factors leading to the habit-forming qualities of tobacco smoking. Nicotine seems to provide both a stimulant and a depressant effect, and it is likely that the effect it has at any time is determined by the mood of the user, the environment and the circumstances of use. However, in order to receive the nicotine via a tobacco product an individual is exposed to a large number of extremely unhealthy and/or toxic substances that have made smoking one of the major preventable causes of negative health events and death in the United States and many other countries around the world. The mechanism for nicotine cravings are nicotinic receptors in the brain. These receptors mediate normal communication between nerve cells in the brain. It is believed that continued exposure to nicotine creates more nicotinic receptors that in turn create a greater desire to a source of nicotine. Unfortunately that source is most often tobacco. While not desirable to a healthy life style, the negative effects of nicotine are only a very small fraction of the negative effects caused by smoking when compared to the negative effects caused by the other substances that enter a person's body via tobacco usage.

Today, many people attempting to quit smoking have turned to substitute products containing nicotine. To date, non-nicotine products have not demonstrated a proven record of being able to consistently be an effective alternative to smoking or other tobacco products because the non-nicotine products lack the ability to satisfy the signature cravings of the brain's nicotinic receptors.

Nicotine replacement therapy is, to use an old military term, divide and conquer. The goal is if a person's nicotine craving can be satisfied without all the unhealthy or toxic substances of tobacco then only the reduction of nicotine intake will remain as an obstacle to a healthy lifestyle. However, a problem with nicotine replacement therapy, in its current form, with currently available products (e.g., Nicotine gum, lozenges, inhalers and skin patches), is that current nicotine replacement therapy addresses only a portion of the problem.

Doctors have long recommended that individuals attempting to quit smoking should consume large amounts of fluids. Fluid consumption in itself is necessary for individual health. Additionally, persons who stop smoking often misinterpret cravings for a cigarette as hunger, which frequently results in weight gain. Drinking fluids helps suppress the cravings for a cigarette and helps curb weight gain. Therefore, there is a need to address the individual's physiological needs (i.e., their cravings).

Long time smokers are used to having the hand they used to hold a cigarette kept busy by the repetitive movement of that hand towards and away from their mouth while smoking. Thus, smokers develop a psychological habit involving hand motion. This is one example of a physical habit a smoker performs without even being fully aware that the physical habit is a part of their overall smoking habit. Nicotine replacement therapy fails to address this aspect of the smoking habit. Therefore, there is a need to keep the individual's hands busy while they are quitting smoking just as much as there is a need to meet their nicotine needs, even if the individual is using a nicotine replacement product.

However, in addition to these two components of the attempt to quit smoking, there is still a third component that historically has presented a major hurdle, especially to women. That third component is weight gain. Gaining weight has long been associated with a cessation of or reduction in smoking. This is a very real yet simple problem and there are two reasons for its occurrence. One reason is the hand-to-mouth action previously discussed and the second reason is that it is common to misinterpret nicotine cravings for food cravings. Normally, a person who smokes would reach for a cigarette, but when this option is taken away, food fills the need or craving and weight gain results since eating also involves hand motion towards and away from the mouth.

In light of the foregoing, an especially effective cigarette alternative is the nicotine or nicotine-alternative beverage. In addition to the advantages listed above, the nicotine or nicotine-alternative beverage also has a built-in protection against nicotine abuse, because any nicotine concentration in the beverage can be adjusted so that an individual will reach his or her fluid intake limit before consuming a dangerous amount of nicotine.

As noted above, the cigarette-rolling machine enabled a huge increase in cigarette production as well as a substantial decrease in production cost, thereby allowing the cigarette to become an extremely popular product. The object of the present invention is to have a similar effect on the nicotine or nicotine-alternative beverage market through a method which allows for increased beverage production as well as decreased production costs. This will result in cigarette nicotine-alternative beverages becoming more available, less expensive, and more popular with consumers.

It is well known that nicotine is a potent substance. Very small amounts of nicotine can produce noticeable effects in adult humans. In fact, the amount of nicotine appropriate for human consumption in a single use, i.e. the amount required to effectively serve as a single cigarette substitute, is too small to be accurately measured in a conventional beverage production setting. The equipment is not sufficiently precise and any error in measurement could mean lethal results for consumers of the beverage.

It is possible to measure nicotine into single-serving or multi-serving amounts by utilizing precision equipment. However, such equipment is expensive and normally unavailable in conventional beverage production settings. In order to be an attractive substitute for cigarettes, a nicotine beverage must be relatively inexpensive. The use of precision measuring equipment will result in higher-priced nicotine beverages, which will be less attractive to individuals attempting to quit smoking.

However, as outlined above, nicotine is a dangerous substance which should be eliminated from entering the body as much as possible, as quickly as possible. In short, there is a need for a substance as part of nicotine replacement therapy that mimics nicotine without being nicotine.

Accordingly, there is a need for a nicotine-alternative composition and a method of inexpensively producing a composition containing a precise amount of a nicotine-alternative appropriate for a single use by a single individual. Such a method should not require the use of high-precision measuring equipment, but should instead utilize equipment already found in conventional beverage production settings. Additionally, to increase marketability, such a method should be capable of yielding a variety of compositions, including liquids, solids, tablets, pills, and powders. There is a further need for a way to reduce the amount of nicotine entering the body. There is an additional need for a way to meet the psychological, as well as physiological, needs that define the smoking habit. The present invention fulfills these needs and provides other related advantages.

### SUMMARY OF THE INVENTION

The present invention resides in a method for producing a composition containing a precise amount of a nicotine-alternative appropriate for a single use by a single individual. The method utilizes equipment normally found in conventional beverage production settings, and functions by diluting the nicotine-alternative into one or more successive intermediate solutions before yielding the final composition to be mixed into a single-serving beverage. This method of successively diluting the nicotine-alternative eliminates the need for expensive, high-precision measurement equipment. As a result, nicotine-alternative beverages will be less expensive to produce and will have the potential to reach a larger market. The present invention further resides in a method for producing a composition containing a precise amount of nicotine, if any, and a nicotine replacement that delivers the performance of a greater amount of nicotine than the amount of nicotine actually utilized in the beverage, by using a nicotine-alternative that mimics nicotine and the effects nicotine has on the body in order to reduce an individual's use of tobacco products.

More particularly, the present invention comprises a method for producing a consumable nicotine-alternative composition including the steps of measuring a quantity of a cigarette nicotine-alternative alkaloid and/or a larger quantity of lobeline, and diluting the quantities of cigarette nicotine-alternative alkaloid and/or lobeline into one or more successive intermediate solutions, a last of which constitutes a final solution. The final solution is apportioned so that each portion contains a precise quantity of cigarette nicotine-alternative alkaloid and/or lobeline appropriate for consumption in a single use by a single person, and each portion is introduced into a separate single-serving or multi-serving dispenser.

In this regard, each successive intermediate solution has a lower cigarette nicotine-alternative alkaloid and/or lobeline concentration than that of the solution immediately preceding it. Moreover, a cigarette nicotine-alternative alkaloid and/or lobeline concentration of between 0.00005% and 0.9% by volume is attained in each portion

At least one of the portions in the single-serving or multi-serving dispenser is introduced into a beverage of equal or greater portion size by a consumer.

The cigarette nicotine-alternative alkaloid is a non-naturally occurring material or it can be derived from a variety of tobacco. The cigarette nicotine-alternative alkaloid can therefore be nicotine. A nicotine concentration of between 0.00005% and 0.9% by volume is attained in the beverages after addition of each portion thereto.

The primary component of the beverage comprises water, carbonated water, natural juice, and/or alcohol. Each of the portions is introduced into a beverage prior to sale or distribution.

Additional features and advantages of the present invention will become apparent from the following more detailed description.

### DETAILED DESCRIPTION OF THE PREFERRED EMBODIMENTS

The present invention resides in a novel composition and a method for producing a composition containing a precise amount of nicotine and/or a nicotine-alternative appropriate for use by an individual. The nicotine composition may be introduced into a single-serving beverage container in a production setting, or it may be packaged in a single-use dispenser and sold to consumers. Regardless of how the composition is made available, the method of producing the composition remains the same and is the subject of the present invention.

A nicotine beverage satisfies the habitual smoker's need to keep their hands occupied; a failure of which can result in a return to smoking since the desire to perform this involuntary action is nearly as strong as the desire for nicotine. Fortunately, this is an action that can be easily duplicated by holding a glass, cup or bottle, the repetitive hand to mouth action can be reenacted at the same time the chemical cravings are being satisfied. Also, drinking fluids incorporates the same hand-to-mouth motion associated with smoking, which may be psychologically helpful to persons attempting to quit smoking.

Normally a person who smokes would reach for a cigarette, but when this option is taken away, food fills the need or craving and weight gain results since eating also involves hand motion towards and away from the mouth. Again, it is the fluid (e.g., water) in the beverage, not the nicotine that will confront this dilemma. By drinking the beverage, the shear bulk of the fluid will fill a person up and thereby mask the phantom hunger pains. By addressing these needs with fluid, the cravings are being filled with little or no fat and little or no calories which should translate into very little or no weight gain.

Nicotine can be derived from a variety of tobacco plant. In addition to the tobacco plant, nicotine is also found in lower quantities in other members of the Solanaceae (nightshade) family, which includes tomato, potato, eggplant (aubergine), and green pepper. Nicotine alkaloids are also found in the leaves of the coca plant. A synthetic nicotine can also be derived from a non-naturally occurring material.

In one embodiment, the method involves diluting a known, large quantity of nicotine into successive intermediate solutions, whereby the nicotine concentration is progressively reduced. For example, it is known that a cigarette contains roughly 1 to 4 mg of nicotine. Therefore, a nicotine beverage ought to contain an equivalent amount. Since 1 mg of nicotine is too small to be accurately measured in a conventional beverage production setting, the method of the present invention may be employed, for example, as follows: First, a relatively large quantity of nicotine (one easily measurable in a beverage production setting) is mixed into a measured quantity of water, or other solvent, to produce a first intermediate solution. The nicotine concentration in this first intermediate solution may still be too high for human consumption. In that case, the first intermediate solution is then divided into a number of equal portions. Each portion is mixed into its own, separate quantity of water, yielding a number of second intermediate solutions. The nicotine concentrations in each of these second intermediate solutions should be equal to one another, and substantially lower than the nicotine concentration in the first intermediate solution.

This process of diluting successive intermediate solutions will eventually yield a final solution having the precise nicotine concentration desired. The number of dilutions and intermediate solutions required will depend on the desired final nicotine concentration, the size of the available containers for dilution and mixing, and the precision of the available measuring equipment. It is quite possible that the first intermediate solution will contain the desired nicotine concentration.

The final nicotine composition may be introduced into single-serving or multi-serving beverage containers, such as cans or bottles, in a production setting, or it may be packaged in single-use or multi-use dispensers to be mixed into beverages later. In the latter case, the final nicotine composition may even be evaporated to yield the desired quantity of nicotine, which may then be mixed with a water-soluble powder filler and sealed in a packet. Such a packet would contain an appropriate amount of nicotine for a single serving and could be opened and its contents mixed into any beverage of choice. A nicotine concentration of between 0.00005% and 0.9% by volume is attained in the beverages after addition of each portion thereto.

It should be noted that while water is specifically mentioned in describing the dilution process, the method of the present invention may employ a wide range of substances as a solvent. It should also be noted that the method of the present invention may be manipulated to yield a final nicotine composition that is a liquid, a solid, a tablet, a pill, a powder, or any other desired form.

In another embodiment, the above described method can be modified to lower the amount of and/or completely eliminate the use of nicotine in the composition. In the final tally, the physiological and psychological aspects of the smoking habit that needed to be satisfied in order to assist an individual quitting smoking are met by the nicotine beverage described above. However, once the attributes of a nicotine beverage are established, the next priority is to satisfy an individual's nicotine desires but with less actual nicotine than needed to satisfy the individual's nicotine desires.

A substance is needed that mimics nicotine without actually being nicotine is needed. As mentioned above, nicotine is a pyrrolidine-like alkaloid. An alkaloid is a nitrogenous organic molecule that has a pharmacological effect on humans and animals. The name derives from the word alkaline; originally, the term was used to describe any nitrogen-containing base (an amine in modern terms). Most alkaloids have a very bitter taste. Other alkaloids in the Pyrrolidine group to which nicotine belongs are hygrine and cuscohygrine.

Nature provides a substance that mimics nicotine in the form of lobelia through its active ingredient, lobeline. Lobeline is another plant alkaloid which acts in some of the same ways as nicotine. Lobeline is extracted from the herb and seeds of Lobelia inflata, (Indian tobacco), a plant found in Canada and the U.S. and has an alkaloid content of 0.63%. Lobelia's other names are “wild tobacco,” “emetic herb,” “asthma weed,” and “bladder bod,” which provide a hint as to some of the other effects of nicotine on the body. Although it is similar in nature, lobeline is not as potent as nicotine. However, lobeline acts on nicotinic receptors in the body and readily crosses both through the blood and placental barriers. While lobelia's main alkaloid, lobeline, has effects similar to nicotine, lobeline lacks the addictive nature of nicotine. Lobelia, through lobeline, comes close to replicating the effects of nicotine but it is not an exact alternative as it is not as potent as nicotine. However, increasing the amount of lobelia used can increase lobelia's (i.e., lobeline's) effectiveness potential. A positive point in favor of lobelia (i.e., lobeline) is that lobelia creates these near nicotine effects without creating more nicotinic receptors in the human brain.

Cytisine is a toxic pyridine-like alkaloid. Pharmacologically, cytisine exhibits similar effects to nicotine due to the structural similarity of the two molecules. In large doses, cytisine can interfere with respiration and become fatal, just as nicotine can. Fortunately, unlike nature, human beings are capable of combining different substances to achieve a desired result or need. Nicotine is the source of “the edge” that people who smoke describe as that thing which compels them to use tobacco. Lobelia, as well as the other substances mentioned above, comes close to being able to mimic the effects that smokers crave. Therefore, the effects of a given amount of nicotine are able to be replicated with a lower nicotine content than the given amount of nicotine by combining a lower amount of nicotine (than the aforementioned given amount) with an amount of lobelia, cytosine or the like that is larger than the lower amount of nicotine. Further manipulation can reduce and/or eliminate the use of nicotine altogether with only lobeline being used. Additionally, nicotine can be replaced by another alkaloid (e.g., cytisine) that could be used alone or in combination with lobelia (i.e., lobeline).

A method for producing a composition containing a precise amount of nicotine and/or nicotine-alternative appropriate for use by an individual involves diluting a known quantity of a cigarette nicotine-alternative alkaloid and/or a larger quantity of lobeline. The cigarette nicotine-alternative alkaloid can include, without limitation, nicotine or one of the substances that mimics nicotine, such as those discussed above.

The quantity of the cigarette nicotine-alternative alkaloid and/or the larger quantity of lobeline are diluted into successive intermediate solutions, whereby the cigarette nicotine-alternative alkaloid and/or lobeline concentration is progressively reduced. For example, it is known that a cigarette contains roughly 1 to 4 mg of nicotine. Therefore, a reduced nicotine beverage ought to contain an amount of the cigarette nicotine-alternative alkaloid and/or lobeline that would achieve a similar effect. Alternatively, the cigarette nicotine-alternative can be a substance that mimics nicotine without itself being an alkaloid. Since the cigarette nicotine-alternative alkaloid and/or lobeline equivalent of 1 mg of nicotine is too small to be accurately measured in a conventional beverage production setting, the method of the present invention may be employed, for example, as follows: First, a relatively large quantity of cigarette nicotine-alternative alkaloid and/or lobeline (one easily measurable in a beverage production setting) is mixed into a measured quantity of water, or other solvent, to produce a first intermediate solution. The cigarette nicotine-alternative alkaloid and/or lobeline concentration in this first intermediate solution may still be too high for human consumption. In that case, the first intermediate solution is then divided into a number of equal portions. Each portion is mixed into its own, separate quantity of water, yielding a number of second intermediate solutions. The cigarette nicotine-alternative alkaloid and/or lobeline concentrations in each of these second intermediate solutions should be equal to one another, and substantially lower than the cigarette nicotine-alternative alkaloid and/or lobeline concentration in the first intermediate solution. Each successive intermediate solution has a lower cigarette nicotine-alternative alkaloid and/or lobeline concentration than that of the solution immediately preceding it.

This process of diluting successive intermediate solutions will eventually yield a final solution having the precise cigarette nicotine-alternative alkaloid and/or lobeline concentration desired. The number of dilutions and intermediate solutions required will depend on the desired final cigarette nicotine-alternative alkaloid and/or lobeline concentration, the size of the available containers for dilution and mixing, and the precision of the available measuring equipment. It is quite possible that the first intermediate solution will contain the desired cigarette nicotine-alternative alkaloid and/or lobeline concentration. The final solution is apportioned so that each portion contains a precise quantity of cigarette nicotine-alternative alkaloid and/or lobeline appropriate for consumption in a single use by a single person. The goal is to attain a cigarette nicotine-alternative alkaloid and/or lobeline concentration of between 0.00005% and 0.9% by volume in each portion. Thus, one particular end result in each portion could be a liquid composition including nicotine or an alkaloid having a similar direction of activity, content of between 0.00005% and 0.9%, in conjunction with a greater amount of lobelia and/or the active ingredient of lobelia (i.e., lobeline).

The final cigarette nicotine-alternative alkaloid and/or lobeline composition for each portion may be introduced into separate single-serving or multi-serving beverage dispensers like a container, such as cans or bottles, in a production setting, or it may be packaged in separate single-use or multi-use dispensers to be mixed into beverages later. The dispensers may be in various forms including, without limitation, an eyedropper, an at least partially porous pouch (similar to a tea bag), a packet, a capped tube, a dissolvable pill or tablet or the like.

At least one of the portions in the dispensers is introduced into a beverage of equal or greater portion size by a consumer. Each of the portions may be introduced into a beverage prior to sale or distribution. In the case of the separate single-use dispensers to be mixed into beverages later, the final cigarette nicotine-alternative alkaloid and/or lobeline composition may even be evaporated to yield the single-serving quantity of the cigarette nicotine-alternative alkaloid and/or lobeline contained therein, which may then be mixed with a water-soluble powder filler and sealed in a packet. Such a packet would contain an appropriate amount of nicotine-equivalent cigarette nicotine-alternative alkaloid and/or lobeline for a single serving and could be opened and its contents mixed into any beverage of choice. As mentioned above, it should be noted that while water is specifically mentioned in describing the dilution process, the method of the present invention may employ a wide range of substances as a solvent. It should also be noted that the method of the present invention may be manipulated to yield a final cigarette nicotine-alternative alkaloid and/or lobeline composition that is a liquid, a solid, a tablet, a pill, a powder, or any other desired form.

The preceding paragraphs have explained the reasoning for the usage of nicotine alone in beverage form, a combination of nicotine and lobelia (via lobeline) in beverage form and a combination of a cigarette nicotine-alternative alkaloid and/or a larger quantity of lobeline in beverage form. However, as a visit to any store's beverage section will show, consumers have a wide variety of opinions upon which type or flavor of beverage they prefer. It would be a travesty to offer a product that presents so much potential to relieve the negative health events caused by smoking only to have it shunned by a segment of the market for the simple reason that its form or flavor was unpalatable. Therefore, while water is described herein, it is recognized that this formulation may not appeal to the entire targeted market segment. As such, a wide variety of beverages, including some that may include an alcoholic content, are noted as being important to conveying this concept to the largest possible segment of people who could benefit from its usage. Thus, a primary component of the beverage can comprise water, carbonated water, natural fruit juice, and/or alcohol.

Although several embodiments have been described in detail for purposes of illustration, various modifications may be made without departing from the scope and spirit of the invention. Accordingly, the invention is not to be limited, except as by the appended claims.

What is claimed is:

1. A method for producing a consumable nicotine-alternative composition, comprising the steps of:
measuring a quantity of a cigarette nicotine-alternative alkaloid and/or a larger quantity of lobeline;
diluting the quantities of cigarette nicotine-alternative alkaloid and/or lobeline into one or more successive intermediate solutions, a last of which constitutes a final solution;
apportioning the final solution so that each portion contains a precise quantity of cigarette nicotine-alternative alkaloid and/or lobeline (lobelia) appropriate for consumption; and
introducing each portion into a separate single-serving or multi-serving dispenser.

2. The method of claim 1, wherein the cigarette nicotine-alternative alkaloid is a non-naturally occurring material.

3. The method of claim 1, wherein the cigarette nicotine-alternative alkaloid is derived from a variety of tobacco.

4. The method of claim 1, wherein the cigarette nicotine-alternative alkaloid comprises nicotine.

5. The method of claim 1, wherein the apportioning step includes the step of attaining a cigarette nicotine-alternative alkaloid and/or lobeline concentration of between 0.00005% and 0.9% by volume in each portion.

6. The method of claim 1, wherein each successive intermediate solution has a lower cigarette nicotine-alternative alkaloid and/or lobeline concentration than that of the solution immediately preceding it.

7. The method of claim 1, wherein at least one of the portions in the single-serving or multi-serving dispensers is introduced into a beverage of equal or greater portion size by a consumer.

8. The method of claim 7, wherein a nicotine or nicotine-alternative concentration of between 0.00005% and 0.9% by volume is attained in the beverages after addition of each portion thereto.

9. The method of claim 7, wherein a primary component of the beverage comprises water, carbonated water, natural juice, and/or alcohol.

10. The method of claim 1, wherein each of the portions is introduced into a beverage prior to sale or distribution.

11. A method for producing a consumable nicotine-alternative composition, comprising the steps of:
measuring a quantity of a nicotine-alternative and/or a larger quantity of lobeline;
diluting the quantities of nicotine-alternative and/or lobeline into one or more successive intermediate solutions, a last of which constitutes a final solution;
apportioning the final solution so that each portion contains a precise quantity of nicotine-alternative and/or lobeline (lobelia) appropriate for consumption in a single use by a single person;
attaining a nicotine-alternative and/or lobeline concentration of between 0.00005% and 0.9% by volume in each portion; and
introducing each portion into a single-serving or multi-serving dispenser.

12. The method of claim 11, wherein the nicotine-alternative is a non-naturally occurring material.

13. The method of claim 11, wherein the nicotine-alternative is derived from a variety of tobacco.

14. The method of claim 11, wherein each successive intermediate solution has a lower nicotine-alternative and/or lobeline concentration than that of the solution immediately preceding it.

15. The method of claim 11, wherein at least one of the portions in the dispensers is introduced into a beverage of equal or greater portion size by a consumer.

16. The method of claim 15, wherein a nicotine-alternative concentration of between 0.00005% and 0.9% by volume is attained in the beverages after addition of each portion thereto.

17. The method of claim 15, wherein a primary component of the beverage comprises water, carbonated water, natural juice, and/or alcohol.

18. The method of claim 11, wherein each of the portions is introduced into a beverage prior to sale or distribution.

19. A method for producing a consumable nicotine-alternative composition, comprising the steps of:
measuring a quantity of a cigarette nicotine-alternative and/or a larger quantity of lobeline (lobelia);
diluting the quantities of cigarette nicotine-alternative and/or lobeline into one or more successive intermediate solutions, a last of which constitutes a final solution;
apportioning the final solution so that each portion contains a precise quantity of cigarette nicotine-alternative and/or lobeline appropriate for consumption in a single use by a single person, wherein each successive intermediate solution has a lower cigarette nicotine-alternative and/or lobeline concentration than that of the solution immediately preceding it;
attaining a cigarette nicotine-alternative and/or lobeline concentration of between 0.00005% and 0.9% by volume in each portion; and
introducing each portion into a separate single-serving dispenser, wherein at least one of the portions in the single-serving dispensers is introduced into a beverage of equal or greater portion size by a consumer.

20. The method of claim 15, wherein a primary component of the beverage comprises water, carbonated water, natural juice, and/or alcohol.

21. A nicotine-alternative composition, comprising:
a nicotine-alternative alkaloid in a human consumable solution in a concentration of between 0.00005% and 0.9% by volume.

22. The composition of claim 21, wherein the nicotine-alternative alkaloid comprises lobeline (lobelia).

23. The composition of claim 21, wherein the human consumable solution comprises water, carbonated water, natural juice or alcohol.
